(19) United States
(12) Patent Application Publication
(10) Pub. No.: US 2006/0189641 A1
(43) Pub. Date: Aug. 24, 2006

(54) Novel 6-phenlyphenanthridines

(76) Inventor: Beate Schmidt, Allensbach (DE)

Correspondence Address: NATH & ASSOCIATES PLLC, 112 South West Street, Alexandria, VA, 22314, US

(21) Appl. No.: 10/565,526

(22) PCT Filed: Jul. 30, 2004
(86) PCT No.: PCT/EP04/51680

§ 371 Date: Jan. 23, 2006

(30) Foreign Application Priority Data
Jul. 31, 2003 (EP) 03017338.9

(51) Int. Cl. A61K 31/473

(52) U.S. Cl. 514/290

(57) ABSTRACT

Compounds of a certain formula (I) 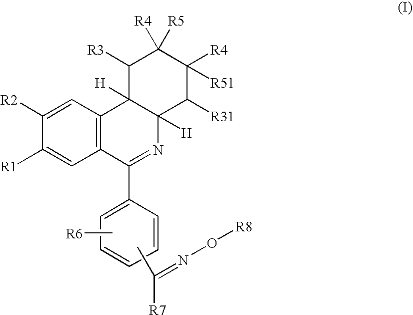 in which R1, R2, R3, R31, R4, R5, R51, R6, R7 and R8 have the meanings indicated in the description, are novel effective PDE4 inhibitors.

### FIELD OF APPLICATION OF THE INVENTION

The invention relates to novel 6-phenylphenanthridines, which are used in the pharmaceutical industry for the production of pharmaceutical compositions.

### KNOWN TECHNICAL BACKGROUND

The international applications WO 97/28131 (=U.S. Pat. No. 6,191,138), WO 97/35854 (=U.S. Pat. No. 6,127,378), WO 99/05113 (=U.S. Pat. No. 6,121,279), WO 99/05111 (=U.S. Pat. No. 6,410,551), WO 00/42018, WO 00/42020, WO 02/05616 and WO 02/06238 describe 6-phenylphenanthridines as PDE4 inhibitors.

### DESCRIPTION OF THE INVENTION

It has now been found that the novel 6-phenylphenanthridines, which are described in greater detail below and differ from the previously known 6-phenylphenanthridines by unanticipated and sophisticated substitution patterns on the 6-phenyl ring, have surprising and particularly advantageous properties.

The invention thus relates to compounds of the formula I, 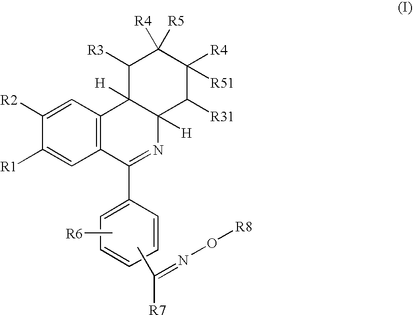
in which
R1 is hydroxyl, 1-4C-alkoxy, 3-7C-cycloalkoxy, 3-7C-cycloalkylmethoxy or completely or predominantly fluorine-substituted 1-4C-alkoxy,
R2 is hydroxyl, 1-4C-alkoxy, 3-7C-cycloalkoxy, 3-7C-cycloalkylmethoxy or completely or predominantly fluorine-substituted 1-4C-alkoxy,
or in which R1 and R2 together are a 1-2C-alkylenedioxy group,
R3 is hydrogen or 1-4C-alkyl,
R31 is hydrogen or 1-4C-alkyl,
or in which R3 and R31 together are a 1-4C-alkylene group,
R4 is hydrogen or 1-4C-alkyl,
R5 is hydrogen,
R51 is hydrogen,
or in which R5 and R51 together represent an additional bond,
R6 is hydrogen, halogen, nitro, 1-4C-alkyl, trifluoromethyl or 1-4C-alkoxy,
R7 is 1-4C-alkyl, 3-7C-cycloalkyl, 3-7C-cycloalkylmethyl, pyridinyl, phenyl or R71-and/or R72-substituted phenyl, wherein
R71 is halogen, hydroxyl, cyano, trifluoromethyl, carboxyl, nitro, 1-4C-alkyl or 1-4C-alkoxy,
R72 is 1-4C-alkoxy, 1-4C-alkyl or halogen,
R8 is hydrogen, phenyl, 1-4C-alkyl, aryloxy-2-4C-alkyl or R9-substituted 1-4C-alkyl, wherein aryloxy is phenoxy or R81-substituted phenoxy, wherein
R81 is halogen or trifluoromethyl,
R9 is phenyl, C(O)N(R91)R92 or R93-substituted phenyl, wherein
R91 is phenyl, aryl-1-4C-alkyl or R911-substituted phenyl, wherein
aryl is phenyl or R81-substituted phenyl,
R911 is 1-4C-alkoxy or completely or predominantly fluorine-substituted 1-4C-alkoxy,
R92 is hydrogen,
or wherein R91 and R92, together and including the nitrogen atom to which both are bound, represent a 1-pyrrolidinyl, 1-piperidinyl, 1-piperazinyl, 4-methylpiperazin-1-yl, 4-phenylpiperazin-1-yl, 1-hexahydroazepinyl or 4-morpholinyl radical,
R93 is nitro, 1-4C-alkyl, 1-4C-alkoxycarbonyl, halogen, trifluoromethyl, 1-4C-alkoxy or completely or predominantly fluorine-substituted 1-4C-alkoxy,
and the salts and the E/Z isomers of these compounds.

1-4C-Alkyl represents a straight-chain or branched alkyl radical having 1 to 4 carbon atoms. Examples which may be mentioned are the butyl, isobutyl, sec-butyl, tert-butyl, propyl, isopropyl and preferably the ethyl and methyl radicals.

1-4C-Alkoxy represents radicals which, in addition to the oxygen atom, contain a straight-chain or branched alkyl radical having 1 to 4 carbon atoms. Examples which may be mentioned are the butoxy, isobutoxy, sec-butoxy, tert-butoxy, propoxy, isopropoxy and preferably the ethoxy and methoxy radicals.

3-7C-Cycloalkoxy represents cyclopropyloxy, cyclobutyloxy, cyclopentyloxy, cyclohexyloxy and cycloheptyloxy, of which cyclopropyloxy, cyclobutyloxy and cyclopentyloxy are preferred.

3-7C-Cycloalkylmethoxy represents cyclopropylmethoxy, cyclobutylmethoxy, cyclopentylmethoxy, cyclohexylmethoxy and cycloheptylmethoxy, of which cyclopropylmethoxy, cyclobutylmethoxy and cyclopentylmethoxy are preferred.

As completely or predominantly fluorine-substituted 1-4C-alkoxy, for example, the 2,2,3,3,3-pentafluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy radicals may be mentioned. “Predominantly” in this connection means that more than half of the hydrogen atoms of the 1-4C-alkoxy radicals are replaced by fluorine atoms.

1-2C-Alkylenedioxy represents, for example, the methylenedioxy [—O—CH₂—O—] and the ethylenedioxy [—O—CH₂—CH₂—O—] radicals.

If R3 and R31 together have the meaning 1-4C-alkylene, the positions 1 and 4 in compounds of the formula I are linked to one another by a 1-4C-alkylene bridge,1-4C-alkylene representing straight-chain or branched akylene radicals having 1 to 4 carbon atoms. Examples which may be mentioned are the radicals methylene [—CH₂—], ethylene [—CH₂—CH₂], trimethylene [—CH₂—CH₂—CH₂—], 1,2-dimethylethylene [—CH(CH₃)—CH(CH₃)—] and isopropylidene [—C(CH₃)₂—].

3-7C-Cycloalkyl represents cyclopropyl, cyclobutyl, cyclopentyl, cyclohexyl and cycloheptyl, of which cyclopropyl, cyclobutyl and cyclopentyl are preferred.

3-7C-Cycloalkylmethyl represents a methyl radical which is substituted by one of the abovementioned 3-7C-cycloalkyl radicals. Preferably, the 3-5C-cycloalkylmethyl radicals cyclopropylmethyl, cyclobutylmethyl and cyclopentylmethyl may be mentioned.

1-4C-Alkoxycarbonyl represents a carbonyl group to which one of the abovementioned 1-4C-alkoxy radicals is bonded. Examples which may be mentioned are the methoxycarbonyl [CH₃O—C(O)—] and the ethoxycarbonyl [CH₃CH₂O—C(O)—] radicals.

R9-substituted 1-4C-alkyl represents one of the abovementioned 1-4C-alkyl radicals, which is substituted by one of the radicals represented by R9. Examples which may be mentioned are the R9-substituted ethyl radicals and, preferably, the R9-substituted methyl radicals.

Aryl represents a phenyl or a R81-substituted phenyl radical.

Aryl-1-4C-alkyl represents one of the abovementioned 1-4C-alkyl radicals, which is substituted by one of the abovementioned aryl radicals. Examples which may be mentioned are the arylethyl and the arylmethyl radicals.

Aryloxy represents a phenoxy or a R81-substituted phenoxy radical.

Aryloxy-2-4C-alkyl represents a 2-4C-alkyl radical, which is substituted by one of the abovementoned aryloxy radicals. Preferably, the 2-aryloxyethyl radical is to be mentioned.

Halogen within the meaning of the invention is bromine, chlorine or fluorine.

Pyridinyl within the meaning of the invention is pyridin-2-yl, pyridin-3-yl or pyridin-4-yl.

The substituents R6 and —C(R7)═N—O—R8 of compounds of the formula I can be attached in the ortho, meta or para position with respect to the binding position in which the 6-phenyl ring is bonded to the phenanthridine ring system. Preference is given to compounds of the formula I, in which R6 is hydrogen and —C(R7)═N—O—R8 is attached in the meta or in the para position.

The person skilled in the art knows that compounds comprising a non-ring C═N double bond can exist in two stereoisomeric forms denoted according common practice in stereochemistry as Z/E isomers. With respect to the oxime C═N double bond, the invention thus relates to any of the possible Z/E isomers and mixtures thereof.

Possible salts for compounds of the formula I—depending on substitution—are all acid addition salts or all salts with bases. Particular mention may be made of the pharmacologically tolerable salts of the inorganic and organic adds and bases customarily used in pharmacy. Those suitable are, on the one hand, water-insoluble and, particularly, water-soluble acid addition salts with acids such as, for example, hydrochloric acid, hydrobromic acid, phosphoric acid, nitric add, sulfuric acid, acetic acid, citric acid, D-gluconic acid, benzoic acid, 2-(4-hydroxybenzyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, it being possible to employ the acids in salt preparation—depending on whether a mono-or polybasic add is concerned and depending on which salt is desired—in an equimolar quantitative ratio or one differing therefrom.

On the other hand, salts with bases are also suitable. Examples of salts with bases which may be mentioned are alkali metal (lithium, sodium, potassium) or calcium, aluminum, magnesium, titanium, ammonium, meglumine or guanidinium salts, where here too the bases are employed in salt preparation in an equimolar quantitative ratio or one differing therefrom.

Pharmacologically intolerable salts which can initially be obtained, for example, as process products in the preparation of the compounds according to the invention on an industrial scale are converted into pharmacologically tolerable salts by processes known to the person skilled in the art.

It is known to the person skilled in the art that the compounds according to the invention and their salts, when they are isolated, for example, in crystalline form, can contain various amounts of solvents. The invention therefore also comprises all solvates and in particular all hydrates of the compounds of the formula I, and also all solvates and in particular all hydrates of the salts of the compounds pounds of the formula I.

Compounds of the formula I to be emphasized are those in which
R1 is 1-2C-alkoxy, 3-5C-cycloalkoxy, 3-5C-cycloalkylmethoxy or completely or predominantly fluorine-substituted 1-2C-alkoxy,
R2 is 1-2C-alkoxy, 3-5C-cycloalkoxy, 3-5C-cycloalkylmethoxy or completely or predominantly fluorine-substituted 1-2C-alkoxy,
R3 is hydrogen,
R31 is hydrogen;
R4 is hydrogen or 1-2C-alky,
R6 is hydrogen,
R51 is hydrogen,
or in which R5 and R51 together represent an additional bond,
R6 is hydrogen,
R7 is 1-4C-alkyl or phenyl,
R8 is hydrogen, phenyl, 1-4C-alkyl, aryloxy-2-4C-alkyl or R9-substituted 1-2C-alkyl, wherein
aryloxy is phenoxy or R81-substituted phenoxy, wherein
R81 is halogen or trifluoromethyl,
R9 is phenyl, C(O)N(R91)R92 or R93-substituted phenyl, wherein
R91 is aryl-1-2C-alkyl or R911-substituted phenyl, wherein
aryl is phenyl or R81-substituted phenyl,
R911 is 1-4C-alkoxy or completely or predominantly fluorine-substituted 1-2C-alkoxy,
R92 is hydrogen,
or wherein R91 and R92, together and including the nitrogen atom to which both are bound, represent a 4-methylpiperazin-1-yl, 4-phenylpiperazin-1-yl or 4-morpholinyl radical,
R93 is nitro, 1-4C-alkoxycarbonyl, halogen, trifluoromethyl, 1-4C-alkoxy or completely or predominantly fluorine-substituted 1-2C-alkoxy,
and the salts and the E/Z isomers of these compounds.

Compounds of the formula I to be more emphasized are those in which
R1 is methoxy,
R2 is methoxy,
R3, R31, R4, R5 and R51 are hydrogen,
R6 is hydrogen,
R7 is 1-2C-alkyl or phenyl,
R8 is hydrogen, phenyl, methyl, ethyl, isobutyl, aryloxyethyl or R9-substituted methyl, wherein
aryloxy is phenoxy or R81-substituted phenoxy, wherein
R81 is trifluoromethyl,
R9 is phenyl, C(O)N(R91)R92 or R93-substituted phenyl, wherein
R91 is arylmethyl or R911-substituted phenyl, wherein
aryl is chlorine-substituted phenyl,
R911 is methoxy.
R92 is hydrogen,
or wherein R91 and R92, together and including the nitrogen atom to which both are bound, represent a 4-phenylpiperazin-1-yl or 4-morpholinyl radical,
R93 is nitro, methoxycarbonyl, chloro, fluoro, trifluoromethyl or methoxy,
and the salts and the E/Z isomers of these compounds.

Compounds of the formula I to be in particular emphasized are those in which either
R1 is methoxy,
R2 is methoxy,
R3, R31, R4, R5 and R51 are hydrogen,
R6 is hydrogen,
R7 is methyl,
R8 is hydrogen, phenyl, methyl, ethyl, isobutyl, phenoxyethyl, 3-trifluoromethylphenoxyethyl or R9-substituted methyl, wherein
R9 is phenyl, 4-nitrophenyl, 4-methoxycarbonylphenyl, 4-chlorophenyl, 3-chlorophenyl, 2-chlorophenyl, 4-fluorophenyl, 3-fluorophenyl, 4-trifluoromethylphenyl, 3-trifluoromethylphenyl, 4-methoxyphenyl, 2-methoxyphenyl or C(O)N(R91)R92, wherein
R91 is 2-methoxyphenyl or 4-chlorobenzyl,
R92 is hydrogen,
or wherein R91 and R92, together and including the nitrogen atom to which both are bound, represent a 4-phenylpiperazin-1-yl or 4-morpholinyl radical;
or
R1 is methoxy,
R2 is methoxy,
R3, R31, R4, R5 and R51 are hydrogen,
R6 is hydrogen,
R7 is phenyl,
R8 is hydrogen;
and the salts and the E/Z isomers of these compounds.

A special embodiment of the compounds of the present invention include those compounds of the formula I in which R1 and R2 are 1-2C-alkoxy.

Another special embodiment of the compounds of the present invention include those compounds of the formula I in which R1 and R2 are 1-2C-alkoxy and R3, R31, R4, R5 and R51 are hydrogen.

A further special embodiment of the compounds of the present invention include those compounds of the formula I in which R1 and R2 are 1-2C-alkoxy and R3, R31, R4, R5, R51 and R6 are hydrogen.

Still a further special embodiment of the compounds of the present invention include those compounds of the formula I in which R1 and R2 are 1-2C-alkoxy and R3, R31, R4, R5, R51 and R6 are hydrogen and R7 is methyl.

Still a further special embodiment of the compounds of the present invention include those compounds of the formula I in which R1 and R2 are 1-2C-alkoxy and R3, R31, R4, R5, R51 and R6 are hydrogen and R7 is phenyl.

The compounds of the formula I are chiral compounds having chiral centers at least in positions 4a and 10b and, depending on the meaning of the substituents R3, R31, R4, R5 and R51, further chiral centers in the positions 1, 2, 3 and 4.

Numbering:

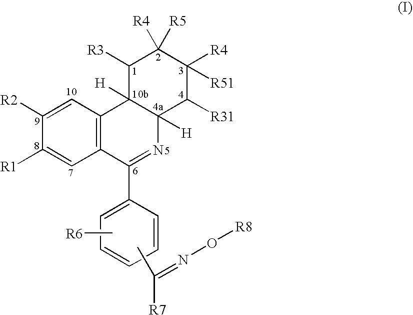

The invention therefore comprises all conceivable stereoisomers in pure form as well as in any mixing ratio.

Preferred compounds of the formula I are those in which the hydrogen atoms in positions 4a and 10b are in the cis position relative to one another. The pure cis diastereomers, the pure cis enantiomers and their mixtures in any mixing ratio and including the racemates are more preferred in this context. Particularly preferred in this connection are those compounds of the formula I which have, with respect to the positions 4a and 10b, the same configuration as shown in the formula I*:

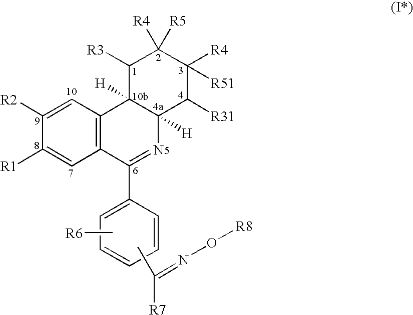

If, for example in compounds of the formula I* R3, R31, R4, R5 and R51 have the meaning hydrogen, then the configuration—according the rules of Cahn, Ingold and Prelog—is R in the position 4a and R in the position 10b.

The enantiomers can be separated in a manner known per se (for example by preparation and separation of appropriate diastereoisomeric compounds). For example, an enantiomer separation can be carried out at the stage of the starting compounds of the formula V in which R1, R2, R3, R31, R4, R5 and R51 have the meanings indicated above.

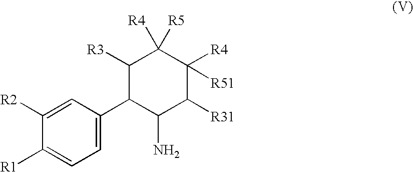

Separation of the enantiomers can be carried out, for example, by means of salt formation of the racemic compounds of the formula V with optically active acids, preferably carboxylic acids, subsequent resolution of the salts and release of the desired compound from the salt. Examples of optically active carboxylic acids which may be mentioned in this connection are the enantiomeric forms of mandelic acid, tartaric acid, O,O′-dibenzoyltartaric acid, camphoric acid, quinic acid, glutamic acid, malic acid, camphorsulfonic acid, 3-bromocamphorsulfonic acid, α-methoxyphenylacetic acid, αmethoxy-αtrifluoromethylphenylacetic acid and 2-phenylpropionic acid. Alternatively enantiomerically pure starting compounds of the formula V can be prepared via asymmetric syntheses. Enantiomerically pure starting compounds as well as enantiomerically pure compounds of the formula I can be also obtained by chromatographic separation on chiral separating columns; by derivatization with chiral auxiliary reagents, subsequent diastereomer separation and removal of the chiral auxiliary group; or by (fractional) crystallization from a suitable solvent

The compounds according to the invention can be prepared, for example, as described in the following examples according to the subsequently specified reaction steps shown in reaction schemes 1 and 2.

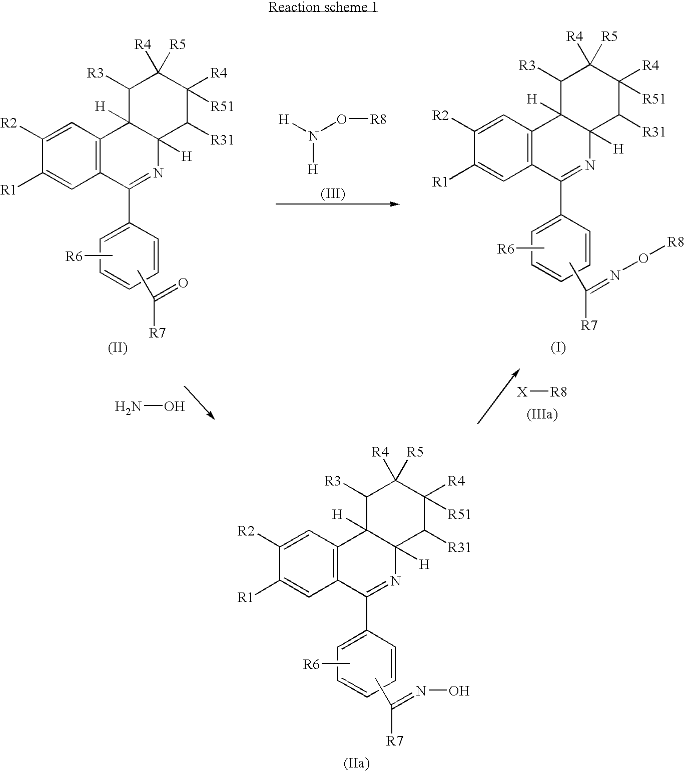

Reaction scheme 1 shows by way of example two alternative synthesis routes for compounds of the formula I, in which R1, R2, R3, R31, R4, R5, R51, R6, R7 and R8 have the meanings indicated above, starting from keto compounds of the formula II, in which R1, R2, R3, R31, R4, R5, R51, R6 and R7 have the meanings indicated above.

One the one hand, said compounds of the formula I are accessible by oxime formation reaction of said compounds of the formula II with compounds of the formula III, in which R8 has the said meaning. Said reaction, can be carried out, for example, as described in the following examples or in a manner known to one of ordinary skill in the art.

On the other hand, said compounds of the formula I can be also obtained in a two step procedure starting from said compounds of the formula II: Firstly, compounds of the formula II are converted with hydroxylamine into corresponding compounds of the formula IIa and then, compounds of the formula IIa obtained are reacted with compounds of the formula IIa, in which R8 has the meanings indicated above and X represents a suitable leaving group, to obtain desired compounds of the formula I. Both of this reactions can be carried out as known to the person skilled in the art.

Compounds of the formula III are either commercially available or can be prepared in an art-known manner.

Compounds of the formula IIIa are known or can be prepared according to known procedures

Compounds of the formula II, in which R1, R2, R3, R31, R4, R5, R51, R6 and R7 have the meanings indicated above, are either known from the international application WO00/42020 or can be prepared similarly or analogously as described herein. Preferably, however, compounds of the formula II are obtained according to those procedures given by way of example in the following examples. For greater detail, a suitable synthesis route for compounds of the formula II is outlined in reaction scheme 2 below. In the first step of said reaction scheme 2 compounds of the formula V, in which R1, R2, R3, R31, R4, R5 and R51 have the meanings given above, are reacted with compounds of the formula VI, in which R6 and R7 have the meanings given above and X represents a suitable leaving group, preferably a chlorine atom, to give compounds of the formula IV, in which R1, R2, R3, R31, R4, R5, R51, R6 and R7 have the abovementioned meanings.

Alternatively, compounds of the formula IV, in which R1, R2, R3, R4, R5, R51, R6, and R7 have the meanings given above, can also be prepared, for example, from compounds of the formula V, in which R1, R2, R3, R31, R4, R5 and R51 have the abovementioned meanings, and compounds of the formula VI, in which R6 and R7 have the abovementioned meanings and X is hydroxyl, by reaction with amide bond linking reagents known to the person skilled in the art. Exemplary amide bond linking reagents known to the person skilled in the art which may be mentioned are, for example, the carbodiimides (e.g. dicyclohexylcarbodiimide or, preferably, 1-ethyl-3-(3-dimethylaminopropyl)carbodiimide hydrochloride), azodicarboxylic acid derivatives (e.g. diethyl azodicarboxylate), uronium salts [e.g. O-(benzotriazol-1-yl)-N,N,N′,N′-tetramethyluronium tetrafluoroborate or O-(benzotriazol-1yl)-N,N,N′,N′-tetramethyl-uronium-hexafluorophosphate] and N,N′-carbonyl-dilimidazole. In the scope of this invention preferred amide bond linking reagents are uronium salts and, particularly carbodiimides, preferably, 1-ethyl-3-(3-dimethylaminopropyl)carbodiimide hydrochloride.

Compounds of the formula VI, wherein R6 and R7 have the abovementioned meanings, are either known or can be prepared in a known manner.

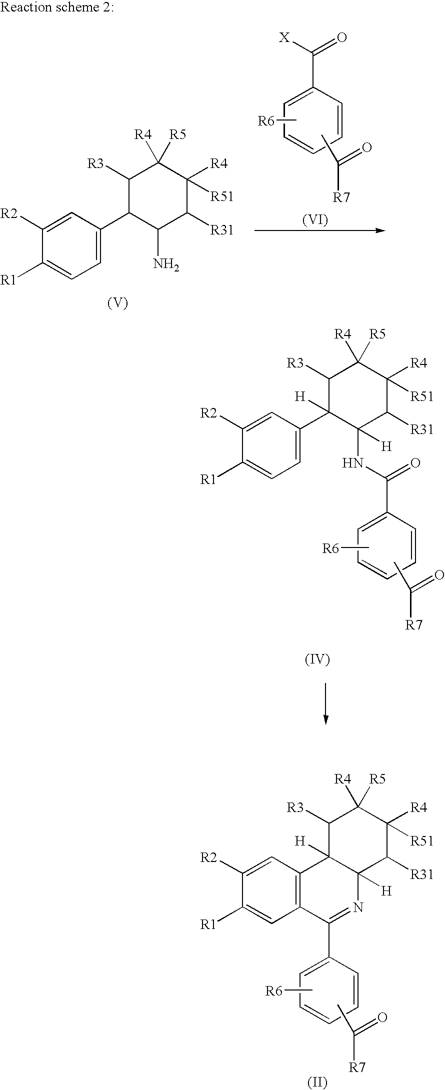

As shown in the next step within reaction scheme 2, compounds of the formula II, in which R1, R2, R3, R31, R4, R5, R51, R6 and R7 have the meanings indicated above, can be obtained by cyclocondensation of corresponding compounds of the formula IV. Said cyclocondensation reaction is carried out in a manner habitual per se to the person skilled in the art or as described by way of example in the following examples, according to Bischler-Napleralski (e.g. as described in J. Chem. Soc., 1956, 4280-4282) in the presence of a suitable condensing agent, such as, for example, polyphosphoric acid, phosphorus pentachloride, phosphorus pentoxide or phosphorus oxychloride, in a suitable inert solvent, e.g. in a chlorinated hydrocarbon such as chloroform, or in a cyclic hydrocarbon such as toluene or xylene, or another inert solvent such as acetonitrile, or without further solvent using an excess of condensing agent, at reduced temperature, or at room temperature, or at elevated temperature or at the boiling temperature of the solvent or condensing agent used.

The preparation of pure enantiomeres of starting compounds of the formula V is predescribed, for example in the international application WO00/42020 or the preparation can be carried out according to the following examples.

It is moreover known to the person skilled in the art that if there are a number of reactive centers on a starting or intermediate compound it may be necessary to block one or more reactive centers temporarily by protective groups in order to allow a reaction to proceed specifically at the desired reaction center. A detailed description for the use of a large number of proven protective groups is found, for example, in “Protective Groups in Organic Synthesis” by T. Greene and P. Wuts (John Wiley & Sons, Inc. 1999, 3^{rd} Ed.) or in “Protecting Groups (Thieme Foundations Organic Chemistry Series N Group” by P. Kocienski (Thieme Medical Publishers, 2000).

The isolation and purification of the substances according to the invention is carried out in a manner known per se, e.g. by distilling off the solvent in vacuo and recrystallizing the resulting residue from a suitable solvent or subjecting it to one of the customary purification methods, such as, for example, column chromatography on suitable support material.

Salts are obtained by dissolving the free compound in a suitable solvent (e.g. a ketone, such as acetone, methyl ethyl ketone or methyl isobutyl ketone, an ether, such as diethyl ether, tetrahydrofuran or dioxane, a chlorinated hydrocarbon, such as methylene chloride or chloroform, or a low molecular weight aliphatic alcohol such as ethanol or isopropanol) which contains the desired acid or base, or to which the desired acid or base is then added. The salts are obtained by filtering, reprecipitating, precipitating with a nonsolvent for the addition salt or by evaporating the solvent. Salts obtained can be converted by alkalization or by acidification into the free compounds, which in turn can be converted into salts. In this way, pharmacologically intolerable salts can be converted into pharmacologically tolerable salts.

Optionally, compounds of the formula I can be converted into their salts, or, optionally, salts of the compounds of the formula I can be converted into the free compounds.

The person skilled in the art knows on the basis of his/her knowledge and on the basis of those synthesis routes, which are shown and described within the description of this invention, how to find other possible synthesis routes for compounds of the formula I. All these other possible synthesis routes are also part of this invention.

The following examples serve to illustrate the invention in greater detail without restricting it. Likewise, further compounds of the formula I, whose preparation is not explicity described, can also be prepared in an analogous manner or in a manner familiar per se to the person skilled in the art using customary process techniques.

In the examples, m.p. stands for meting point, h for hour(s), min for minutes, EF for empirical formula, MW for molecular weight, MS for mass spectrum, M for molecular ion, calc. for calculated, fnd. for found.

The compounds mentioned in the examples and their salts and E/Z isomers are a preferred subject of the invention.

### EXAMPLES

Final Products:

### 1. 1-[4-((4aR,10bR)-8,9-Dimethoxy-1,2,3,4,4a,10b-hexahydrophenanthridin-6-yl}-phenyl]-ethanone oxime

200 mg of (4aR,10bR)-8,9-dimethoxy-6-(4-acetophenyl)-1,2,3,4a,10b-hexahydrophenanthridine (compound A1) and 300 mg of sodium hydrogencarbonate are suspended in 5 ml of ethanol, treated with 230 mg of hydroxylamine hydrochloride and stirred for 1.5 h at room temperature. The reaction mixture is filtered, the filtrate concentrated and the residue chromatographed on silica gel (petroleum ether/ethyl acetate/triethylamine 6:3:1). 72 mg of the the compound are obtained. M.p. 189-192° C.

MS: calc.: C₂₃H₂₆N₂O₃ (378.48) fnd.: [M+1] 379.2

Starting from the appropriate starting compounds A1 or A2 or A3 or A4 described below, the following compounds are obtained in analogy to the procedure as in Example 1 using appropriately O-substituted hydroxylamines as reaction partners.

### 2. 1-[4-((4aR,10bR)-8,9-Dimethoxy-1,2,3,4a,10b-hexahydrophenanthridin-6-yl}phenyl]-1-phenyl-methanone oxime

MS: calc.: C₂₈H₂₈N₂O₃ (440.55) fnd.: [M+1] 441.3

### 3. 1-[4-((4aR,10bR)-8,9-Dimethoxy-1,2,3,4,4a,10b-hexahydrophenanthridin-6-yl}-phenyl]-ethanone O-methyloxime

MS: calc.: C₂₄H₂₈N₂O₃ (392.5) fnd.: [M+1] 393.2

### 4. 1-[4-((4aR,10bR)-8,9-Dimethoxy-1,2,3,4,4a,10b-hexahydrophenanthridin-6-yl}-phenyl]-ethanone O-benzyloxime

MS: calc.: C₃₀H₃₂N₂O₃ (468.6) fnd.: [M+1] 469.2

### 5. 1-[4-((4aR,10bR)-8,9-Dimethoxy-1,2,3,4,4a,10b-hexahydrophenanthridin-6-yl}-phenyl]-ethanone O-(4-nitrobenzyl)-oxime

MS: calc.: C₃₀H₃₁N₃O₅ (513.6) fnd.: [M+1] 514.2

### 6. 1-[4-((4aR,10bR)-8,9-Dimethoxy-1,2,3,4,4a,10b-hexahydrophenanthridin-6-yl}-phenyl]ethanone O-ethyloxime

MS: calc.: C₂₅H₃₀N₂O₃ (406.53) fnd.: [M+1] 407.2

### 7. 1-[4-((4aR,10bR)-8,9Dimethoxy-1,2,3,4,a,10b-hexahydrophenanthridin-6-yl}-phenyl]ethanone O-phenyloxime

MS: calc.: C₂₉H₃₀N₂O₃ (454.57) fnd.: [M+1] 455.0

### 8. 1-[4-((4aR,10bR)-8,9-Dimethoxy-1,2,3,4,4a,10b-hexahydrophenanthridin-6yl)-phenyl]-ethanone O-isobutyloxime

MS: calc.: C₂₇H₃₄N₂O₃ (434.58) fnd.: [M+1] 435.2

### 9. 4-{1-[4-((4aR,10bR)-8,9-Dimethoxy-1,2,3,4,4a,10b-hexahydrophenanthridin-6yl)-phenyl]ethylideneaminooxymethyl}-benzoic acid methyl ester

MS: calc.: C₃₂H₃₄N₂O₅ (526.64) fnd.: [M+1] 527.3

### 10. 2-{1-[4-((4aR,10bR)-8,9-Dimethoxy,1,2,3,4,4a,10b-hexahydrophenanthridin-6yl)-phenyl]ethylideneaminooxy}-1-(4-phenylpiperazin-1-yl)-ethanone

MS: calc.: C₃₅H₄₀N₄O₄ (580.73) fnd.: [M+1] 581.3

### 11. 2-{1-[4-((4aR,10bR)-8,9-Dimethoxy-1,2,3,4,4a,10b-hexahydrophenanthridin-6-yl)-phenyl]ethylideneaminooxy}-N-(2-methoxyphenyl)-acetamide

MS: calc.: C₃₂H₃₅N₃O₅ (541.65) fnd.: [M+1] 542.3

### 12. N-(4-Chlorobenzyl)-2-{1-[4-((4aR,10bR)-8,9-dimethoxy-1,2,3,4,a,10b-hexahydrophenanthridin-6-yl)-phenyl]-ethylideneaminooxy}-acetamide

MS: calc.: C₃₂H₃₄Cl N₃O₄ (560.1) fnd.: [M+1] 560.3

### 13. 1-]4-((4aR,10bR)-8,9-Dimethoxy-1,2,3,4,4a,10b-hexahydrophenanthridin-6-yl}-phenyl]-ethanone O-(3-chloro-benzyl)-oxime

MS: calc.: C₃₀H₃₁Cl N₂O₃ (503.05) fnd.: [M+1] 503.2

### 14. 1-[4-((4aR,10bR)-8,9-Dimethoxy-1,2,3,4,4a,10b-hexahydrophenanthridin-6-yl}-phenyl]-ethanone O-(3-trifluoromethylbenzyl)-oxime

MS: calc.: C₃₁H₃₁F₃N₂ (536.6) fnd.: [M+1] 537.2

### 15. 1-[4-((4aR,10bR)-8,9-Dimethoxy-1,2,3,4,4a,10b-hexahydrophenanthridin-6-yl}-phenyl]-ethanone O-(2-methoxy-benzyl)-oxime

MS: calc.: C₃₁H₃₄N₂O₄ (498.63) fnd.: [M+1] 499.2

### 16. 1-[4-((4aR,10bR)-8,9-Dimethoxy1,2,3,4,4a,10b-hexahydrophenanthridin-6-yl}-phenyl]-ethanone O-(4-flurobenzyl)-oxime

MS: calc.: C₃₀H₃₁F N₂O₃ (486.69) fnd.: [M+1] 487.2

### 17. 1-[4-((4aR,10bR)-8,9-Dimethoxy-1,2,3,4,4a,10b-hexahydrophenanthridin)-6-yl}-phenyl]-ethanone O-(3-flurobenzyl)-oxime

MS: calc.: C₃₀H₃₁F N₂O₃ (488.59) fnd.: [M+1] 487.2

### 18. 1-[4-((4aR,10bR)-8,9-Dimethoxy-1,2,3,4,4a,10b-hexahydrophenanthridin-6-yl}-phenyl]-ethanone O-(2-chlorobenzyl)-oxime

MS: calc.: C₃₀H₃₁Cl N₂O₃ (503.05) fnd.: [M+1] 503.2

### 19. 1-[4-((4aR,10bR)-8,9-Dimethoxy-1,2,3,4,4a,10b-hexahydrophenanthridin-6-yl)-phenyl]-ethanone O-(2-phenoxyethyl)-oxime

MS: calc.: C₃₁H₄N₂O₄ (498.63) fnd.: [M+1] 499.2

### 20. 1-[4-((4aR,10bR)-8,9-Dimethoxy-1,2,3,4,4a,10b-hexahydrophenanthridin-6-yl}-phenyl]-ethanone O-[2-(3-trifluoromethylphenoxy)-ethyl]-oxime

MS: calc.: C₃₂H₃₃F₃N₂O₄ (566.63) fnd.: [M+1] 567.2

### 21. 1-[4-((4aR,10bR)-8,9-Dimethoxy-1,2,3,4,4a,10b-hexahydrophenanthridin-6-yl}-phenyl]-ethanone O-(4-trifluoromethylbenzyl)-oxime

MS: calc.: C₃₁H₃₁F₃N₂O₃ (536.6) fnd.: [M+1] 537.2

### 22. 1-[4-((4aR,10bR)-8,9-Dimethoxy-1,2,3,4,4a,10b-hexahydrophenanthridin-6-yl)-phenyl]-ethanone O-(4-chlorobenzyl)-oxime

MS: calc.: C₃₀H₃₁Cl N₂O₃ (503.05) fnd.: [M+1] 503.2

### 23. 1-[4-((4aR,10bR)-8,9-Dimethoxy-1,2,3,4,4a,10b-hexahydrophenanthridin-6}-phenyl]-ethanone O-(4-methoxybenzyl)-oxime

MS: calc.: C₃₁H₃₄N₂O₄ (498.63) fnd.: [M+1] 499.3

### 24. 2-{1-[4-((4aR,10bR)-8,9-Dimethoxy-1,2,3,4,4a,10b-hexahydrophenanthridin-6-yl)-phenyl]-ethylideneaminooxy}-1-morpholin-4-yl-ethanone

MS: calc.: C₂₉H35N₃O₅ (505.62) fnd.: [M+1] 506.3

### 25. 1-((4aR,10bR)-8,9-Dimethoxy-1,2,3,4,4a,10b-hexahydrophenanthridin-6-yl}-phenyl]-ethanone oxime

MS: calc.: C₂₃H₂₆N₂O₃ (378.48) fnd.: [M+1] 379.4

### 26. 1-[3-((4aR,10bR)-8,9-Dimethoxy-1,2,3,4,4a,10b-hexahydrophenanthridin-6-yl}-phenyl]-1-phenyl-methanone oxime

MS: calc.: C₂₈H₂₈N₂O₃ (440.55) fnd.: [M+1] 441.3

### 27. 1-[3-((4aR,10bR)-8,9-Dimethoxy-1,2,3,4,4a,10b-hexahydrophenanthridin-6-yl}-phenyl]-ethanone O-benzyloxime

MS: calc.: C₃₀H₃₂N₂O₃ (468.6) fnd.: [M+1] 469.2

### 28. 1-[3-((4aR,10bR)-8,9-Dimethoxy1,2,3,4,4a,10b-hexahydrophenanthridin-6-yl}-phenyl]-ethanone O-methyloxime

MS: calc.: C₂₄H₂₉N₂O₃ (293.5) fnd.: [M+1] 393.2

### 29. 1-[3-(4aR,10bR)-8,9-Dimethoxy-1,2,3,4,4a,10b-hexahydrophenanthridin-6-yl}-phenyl]-ethanone O-(4-chloro-benzyl)oxime

MS: calc.: C₃₀H₃₁Cl N₂O₃ (503.05) fnd.: [M+1] 503.2

### 30. 1-[3-((4aR,10bR)-8,9-Dimethoxy-1,2,3,4,4a,10b-hexahydrophenanthridin-6-yl}-phenyl]-ethanone O-(4-methoxy-benzyl)oxime

MS: calc.: C₃₁H₃₄N₂O₄ (498.63) fnd.: [M+1] 499.2

### 31. 2-{1-[3-((4aR,10bR)-8,9-Dimethoxy-1,2,3,4,4a,10b-hexahydrophenanthridin-6yl)-phenyl]-ethylideneaminooxy}-1-morpholin-4-yl-ethanone

MS: calc.: C₂₉H₃₅N₃O₅ (505.62) fnd.: [M+1] 506.2

### 32. 1-[3-((4aR,10bR)-8,9-Dimethoxy-1,2,3,4,4a,10b-hexahydrophenanthridin-6yl}-phenyl]-ethanone O-(4-nitro-benzyl)-oxime

MS: calc.: C30H31N3O5 (513.6) fnd.: [M+1] 514.2

### 33. 1-[3-((4aR,10bR)-8,9-Dimethoxy1,2,3,4,4a,10b-hexahydrophenanthridin-6)-phenyl]-ethanone O-ethyloxime

MS: calc.: C₂₅H₃₀N₂O₃ (406.53) fnd.: [M+1] 407.2

### 34. 1-[3-((4aR,10bR)-8,9-Dimethoxy1,2,34,4a,10b-hexahydrophenanthridin-6-yl}-phenyl]-ethanone O-phenyloxime

MS: calc.: C₂₉H₃₀N₂O₃ (454.57) fnd.: [M+1] 455.0

### 35. 1-[3-((4aR,10bR)-8,9-Dimethoxy1,2,3,4,4a,10b-hexahydrophenanthridin-6-yl)-phenyl]-ethanone O-isobutyloxime

MS: calc.: C₂₇H₃₄N₂O₃ (434.58) fnd.: [M+1] 435.2

### 36. 4-{1-[3-((4aR,10bR)-8,9-Dimethoxy-1,2,3,4,4a,10b-hexahydrophenanthridin-6yl)-phenyl]-ethylideneaminooxymethyl}-benzolcacidmethylester

MS: calc.: C₃₂H₃₄N₂O₅ (526.64) fnd.: [M+1] 527.2

### 37. 2-{1-[3-((4aR,10bR)-8,9-Dimethoxy-1,2,3,4,4a,10b-hexadrophenanthridin-6yl)-phenyl]-ethylideneaminooxy}-1-(4-phenyl-piperazin-1-yl)-ethanone

MS: calc.: C₃₅H₄₀N₄ O₄ (580.73) fnd.: [M+1] 581.3

### 38. 2-{1-[3-((4aR,10bR)-8,9-Dimethoxy-1,2,3,4,4a,10b-hexadrophenanthridin-6yl)-phenyl]-ethylideneaminooxy}-N-(2-methoxy-phenyl)-acetamide

MS: calc.: C₃₂H₃₅N₃O₅ (541.65) fnd.: [M+1] 542.3

### 39. N-(4-chloro-benzyl)-2-{1-[3-((4aR,10bR)-8,9-Dimethoxy-1,2,3,4,4a,10b-hexahydrophenanthridin-6yl)-phenyl]-ethylideneaminooxy}-acetamide

MS: calc: C₃₂H₃₄Cl N₃O₄ (560.1) fnd.: [M+1] 560.3

### 40. 1-[3-((4aR,10bR)-8,9-Dimethoxy-1,2,3,4,4a,10b-hexahydrophenanthridin-6-yl}-phenyl]-ethanone O-(3-chloro-benzyl)-oxime

MS: calc: C₃₀H₃₁Cl N₂O₃ (503.05) fnd.: [M+1] 503.2

### 41. 1-[3-((4aR,10bR)-8,9-Dimethoxy-1,2,3,4,4a,10b-hexahydrophenanthridin6-yl}-phenyl]-ethanone O-(3-fluoromethyl-benzyl)-oxime

MS: calc.: C₃₁H₃₁F₃N₂O₃ (536.6) fnd.: M+1] 537.2

### 42. 1-[3-((4aR,10bR)-8,9-Dimethoxy,1,2,3,4,4a,10b-hexahydrophenanthridin-6yl)-phenyl]-ethanone O(-3-methoxy-benzyl)-oxime

MS: calc.: C₃₁H₃₄N₂O₄ (498.63) fnd.: [M+1] 499.2

### 43. 1-[3-((4aR,10bR)-8,9-Dimethoxy-1,2,3,4,4a,10b-hexahydrophenanthridin-6-yl)-phenyl]-ethanone O-fluoro-benzyl)-oxime

MS: calc.: C₃₀H₃₁F N₂O₃ (486.59) fnd.: [M+1] 487.2

### 44. 1-[3-((4aR,10bR)-8,9-Dimethoxy-1,2,3,4,4a,10b-hexahydrophenanthridin-6yl}-phenyl]-ethanone O-(3-fluoro-benzyl)-oxime

MS: calc.: C₃₀H₃₁F N₂O₃ (486.59) fnd.: [M+1] 487.2

### 45. 1-'3-((4aR,10bR)-8,9-Dimethoxy-1,2,3,4,4a,10b-hexahydrophenanthridin-6-yl}-phenyl]-ethanone O-(2-chlorobenzyl)-oxime

MS: calc.: C₃₀H₃₁Cl N₂O₃ (503.05) fnd.: [M+1] 503.2

### 46. 1-[3-((4aR,10bR)-8,9-Dimethoxy-1,2,3,4,4a,10b-hexahydrophenanthridinr-6-yl}-phenyl]-ethanone O-(2-phenoxyethyl)oxime

MS: calc.: C₃₁H₃₄N₂O₄ (498.63) fnd.: [M+1] 499.7

### 47. 1-[3-((4aR,10bR)-8,9-Dimethoxy1,2,3,4,4a,10b-hexahydro-phenanthridin-6yl)-phenyl]-ethanone O-[2-(3-trifluoromethyl-phenoxy)-ethyl]-oxime

MS: calc.: C₃₂H₃₃F₃N₂O₄ (566.63) fnd.: [M+1] 567.2

### 48. 1-[3-((4aR,10bR)-8,9-Dimethoxy-1,2,3,4,4a,10b-hexahydrophenanthridin-6yl)-phenyl]-ethanone O-(4-trifluoromethylbenzyl)-oxime

MS: calc.: C₃₁H₃₁F₃N₂O₃ (536.6) fnd.: [M+1] 537.2

Starting Compounds:

### A1. (4aR,10bR)-8,9-Dimethoxy-6-(4-acetophenyl)-1,2,3,4,4a,10b-hexahydrophenanthridine

Compound A1 is prepared from N-[(1R,2R)-2-(3,4-dimethoxyphenyl)cyclohexyl]-4-acetobenzamide (compound B1) analogously as described in Example A2.

EF: C₂₃H₂₅N O₃; MW: 363.46

Elemental analysis: calc.: C, 76.01; H, 6.93; N, 3.85. fnd: C, 75.77; H, 6.98; N, 3.82.

Optical rotation: [α]_{D}²⁰=−97.4° (c=0.2, ethanol)

### A2. (4aR,10bR)-8,9-Dimethoxy-6-(4-benzoylphenyl)-1,2,3,4,4a,10b-hexahydrophenanthridine

7.1 g of N-[(1R,2R)-2-(3,4-dimethoxyphenyl)cyclohexyl[-4-benzoylbenzamide (compound B2) are dissolved in 100 ml of acetonitrile and 5.0 ml of phosphoryl chloride and stirred overnight at 80° C. The reaction mixture is concentrated under reduced pressure and the residue is extracted with said. sodium hydrogencarbonate solution and ethyl acetate. After chromatography on silica gel using petroleum ether (low)/ethyl acetate/triethylamine in the ratio 6/3/1 and concentration of the product fractions, 5.3 g of the title compound are obtained.

EF: C₂₈H₂₇ N O₃; MW: 425.53

Elemental analysis×0.08 H₂O: calc.: C, 78.77; H, 6.64; N, 3.28. fnd: C, 78.55; H, 6.64; N, 3.50.

Optical rotation: [α]_{D}²⁰=−70.6° (c=0.2, ethanol)

### A3. (4aR,10bR)-8,9-Dimethoxy-6-(3-acetophenyl)-1,2,3,4,4a,10b-hexahydrophenanthridine

Compound A3 is prepared from N-[(1R,2R)-2-(3,4-dimethoxyphenyl)cyclohexyl]-3-acetobenzamide (compound B3) analogously as described in Example A2.

M.p. 112.5-114° C.

EF: C₂₃H₂₅N O₃; MW:363.46

Elemental analysis: calc.: C, 76.01; H, 6.93; N, 3.85. fnd.: C, 75.62; H, 6.90; N, 3.83.

Optical rotation: [α]_{D}²⁰=168.7° (c=0.2, ethanol)

### A4. (4aR,10bR)-8,9-Dimethoxy-6-(3-benzoylphenyl)-1,2,3,4,4a,10b-hexahydrophenanthridine

Compound A4 is prepared from N-[(1R,2R)-2-(3,4-dimethoxyphenyl)cyclohexy]-3-benzoylbenzamide (compound B4) analogously as described in Example A2.

EF: C₂₈ H₂₇ N O₃; MW:425.53

Elemental analysis×0.15 H₂O: calc: C, 78.64; H, 6.43; N, 3.27. fnd: C, 78.39; H, 6.58; N, 3.40.

Optical rotation: [α]_{D}²⁰=96.8° (c=0.2, ethanol)

### B1. N-[(1R,2R)-2-(3,4-Dimethoxyphenyl)cyclohexyl]-4-acetobenzamide

Compound B1 is prepared from (1R,2R)-2-(3,4-dimethoxyphenyl)cyclohexylamine (compound C1) analogously as described in Example B2.

M.p.: 129-137° C.

Optical rotation: [α]_{D}²⁰=−180.4° (c=0.2, ethanol)

### B2. N-[(1R,2R)-2-(3,4-Dimethoxyphenyl)cyclhexyl]-4-benzoylbenzamide

4.0 g of (1R,2R)-2-(3,4-dimethoxyphenyl)cyclohexylamine (compound C1) are dissolved in 40 ml of methylene chloride and 10.0 ml of triethylamine. A solution of 4.9 g of benzophenone-4-carbonyl chloride in 100 ml of methylene chloride is added dropwise at RT and the mixture is extracted, after stirring overnight, with 50 ml each of water, 2N hydrochloric acid, satd sodium hydrogencarbonate solution and water again. The organic phase is dried using sodium sulfate and concentrated. 7.78 g of the title compound are obtained as a crystallizing oil.

M.p.: 119-122.5° C.

Optical rotation: [α]_{D}²⁰=−151.7° (c=0.2, ethanol)

### B3. N-[(1R,2R)-2-(3,4-Dimethoxyphenyl)cyclohexyl]-3-acetobenzamide

Compound B3 is prepared from compound C1 analogously as described in Example B2.

Solidifying oil.

Optical Rotation: [α]_{D}=127.1° (c=0.2, ethanol)

### B4. N-[(1R,2R)-2-(3,4-Dimethoxyphenyl)cyclohexyl]-3-benzoylbenzamide

Compound B4 is prepared from compound C1 analogously as described in Example B2.

Oil.

Optical rotation: [α]_{D}=162.9° (c=0.2, ethanol)

### C1. (1R,2R)-2-(3,4-Dimethoxyphenyl)cyclohexylamine

12.0 g of a racemic mixture of (1R,2R)-2-(3,4-dimethoxyphenyl)-cyclohexylamine and (1S,2S)-2-(3,4-dimethoxyphenyl)-cyclohexylamine (compound D1) and 6.2 g of (−)-mandelic acid are dissolved in 420 ml of dioxane and 60 ml of tetrahydrofuran and the solution is stirred overnight at RT. The solid is filtered off with suction, dried, treated with 100 ml of saturated sodium hydrogencarbonate solution and extracted with ethyl acetate. The organic phase is dried using sodium sulfate and concentrated under reduced pressure. 4.8 g of the title compound are obtained of m.p.: 80-81.5° C.

Specific rotation: [α]_{D}²⁰=−58.5° C. (c=1, ethanol).

### D1. Racemic mixture of (1R,2R)-2-(3,4-dimethoxyphenyl)-cyclohexylamine and (1S,2S)-2-(3,4-dimethoxyphenyl)-cyclohexylamine

125 g of a racemic mixture of 1,2-dimethoxy4-((1R,2R)-2-nitrocyclohexy)benzene and 1,2-dimethoxy-4-((1S,2S)-2-nitrocyclohexyl)benzene (compound E1) and 120 g of zinc powder or granules are suspended in 1300 ml of ethanol. 220 ml of acetic acid are added dropwise at boiling heat. The precipitate is filtered off with suction and washed with ethanol, and the filtrate is concentrated under reduced pressure. The residue is taken up in hydrochloric acid and extracted with toluene. The aqueous phase is rendered alkaline using 50% strength sodium hydroxide solution, the precipitate is filtered off with suction and the filtrate is extracted with toluene. The organic phase is dried using sodium sulfate and concentrated. 98 g of the title compound are obtained as a crystallizing oil.

Alternatively:

8.5 g of a racemic mixture of 1,2-dimethoxy-4-((1R,2R)-2-nitrocyclohexyl)benzene and 1,2-dimethoxy-4-((1S,2S)-2-nitrocyclohexyl)benzene (compound E1) are dissolved in 400 ml of methanol and treated at RT with 7 ml of hydrazine hydrate and 2.5 g of Raney nickel in portions in the course of 8 h. After stirring overnight at RT, the reaction mixture is filtered, the filtrate is concentrated and the residue is chromatographed on silica gel using a mixture of toluene/ethyl acetate/triethylamine=4/2/0.5. The title compound is obtained as an oil.

### E1. Racemie mixture of 1,2-dimethoxy-4-((1R,2R)-2-nitrocyclohexyl)benzene and 1,2-dimethoxy-4-((1S,2S)-2-nitrocyclohexyl)benzene

8.4 g of a racemic mixture of 1,2-dimethoxy-4-((1R,2R)-2-nitrocyclohex 4-enyl)benzene and 1,2-dimethoxy-4-((1s,2S)-2-nitrocyclohex-4-enyl)benzene (compound F1) are dissolved in 450 ml of methanol, treated with 2 ml of conc. hydrochloric acid and hydrogenated after addition of 500 mg of 10% strength Pd/C. The reaction mixture is filtered and the filtrate is concentrated. M.p.: 84-86.5° C.

### F1. Racemic mixture of 1,2-dimethoxy-4-((1R,2R)-2-nitroyclohex-4-enyl)benzene and 1,2-dimethoxy-4-((1S,2S)-2-nitrocyclohex-4-enyl)benzene

10.0 g of a racemic mixture of 1,2-dimethoxy-4-((1R,2S)-2-nitrocyclohex-4-enyl)benzene and 1,2-dimethoxy-4-((1s,2R)-2-nitrocyclohex-4-enyl/benzene (compound G1) and 20.0 g of potassium hydroxide are dissolved in 150 ml of ethanol and 35 ml of dimethylformamide. A solution of 17.5 ml of cone. sulfuric acid in 60 ml of ethanol is then added dropwise such that the internal temperature does not exceed 40° C. After stirring for 1 h, the mixture is added to 1 l of ice water, the precipitate is filtered off with suction, washed with water and dried, and the crude product is recrystallized from ethanol. 8.6 g of the title compound of m.p. 82.5-84° C. are obtained.

### G1. Racemic mixture of 1,2-dimethoxy4-((1R,2S)-2-nitrocyclohex-4-enyl)benzene and 1,2-dimethoxy-4-((1S,2R)-2-nitrocyclohex-4-enyl)benzene

50.0 g of 3,4-dimethoxy-ω-nitrostyrene (compound H1) and 1.0 g (9.1 mmol) of hydroquinone are suspended in 200 ml of abs. toluene and treated at −70° C. with 55.0 g (1.02 mol) of liquid 1,3-butadiene. The mixture is stirred at 160° C. for 6 days in an autoclave and then cooled. Some of the solvent is removed on a rotary evaporator, and the resulting precipitate is filtered off with suction and recrystallized in ethanol. M.p.: 113.5-115.5° C.

### H1. 3,4-Dimethoxy-ω-nitrostyrene

207.0 g of 3,4-dimethoxybenzaldehyde, 100.0 g of ammonium acetate and 125 ml of nitromethane are heated to boiling for 3-4 h in 1.0 l of glacial acetic acid. After cooling in an ice bath, the precipitate is filtered off with suction, rinsed with glacial acetic acid and petroleum ether and dried. M.p.: 140-141° C. Yield: 179.0 g.

Commercial Utility

The compounds according to the invention have useful pharmacological properties which make them industrially utillizable. As selective cyclic nucleotide phosphodiesterase (PDE) inhibitors (specifically of type 4), they are suitable on the one hand as bronchial therapeutics (for the treatment of airway obstructions on account of their dilating action but also on account of their respiratory rate- or respiratory drive-increasing action) and for the removal of erectile dysfunction on account of their vascular dilating action, but on the other hand especially for the treatment of disorders, in particular of an inflammatory nature, e.g. of the airways (asthma prophylaxis), of the skin, of the intestine, of the eyes, of the CNS and of the joints, which are mediated by mediators such as histamine, PAF (platelet-activating factor), arachidonic acid derivatives such as leukotrienes and prostaglandins, cytokines, interleukins, chemokines, alpha-, beta- and gamma-interleon, tumor necrosis factor (TNF) or oxygen free radicals and proteases. In this context the compounds according to the invention are distinguished by a low toxicity, a good enteral absorption (high bioavailability), a large therapeutic breadth and the absence of significant side effects.

On account of their PDE-inhibiting properties, the compounds according to the invention can be employed in human and veterinary medicine as therapeutics, where they can be used, for example, for the treatment and prophylaxis of the following illnesses: acute and chronic (in particular inflammatory and allergen-induced) airway disorders of varying origin (bronchitis, allergic bronchitis, bronchial asthma, emphysema, COPD); dermatoses (especially of proliferative, inflammatory and allergic type) such as psoriasis (vulgaris), toxic and allergic contact eczema, atopic eczema, seborrhoeic eczema, Lichen simplex, sunburn, pruritus in the anogenital area, alopecia areata, hypertrophic scars, discold lupus erythematosus, follicular and widespread pyodermias, endogenous and exogenous acne, acne rosacea and other proliferative, inflammatory and allergic skin disorders; disorders which are based on an excessive release of TNF and leukotrienes, for example disorders of the arthritis type (rheumatoid arthritis, rheumatoid spondylitis, osteoarthritis and other arthritic conditions), disorders of the immune system (AIDS, multiple sclerosis), graft versus host reaction, allograft rejections, types of shock (septic shock, endotoxin shock, gram-negative sepsis, toxic shock syndrome and ARDS (adult respiratory distress syndrome)) and also generalized inflammations in the gastrointestinal region (Crohn's disease and ulcerative colitis); disorders which are based on allergic and/or chronic, immunological false reactions in the region of the upper airways (pharynx, nose) and the adjacent regions (paranasal sinuses, eyes), such as allergic rhinitis/sinusitis, chronic rhinitis/sinusitis, allergic conjunctivitis and also nasal polyps; but also disorders of the heart which can be treated by PDE inhibitors, such as cardiac insufficiency or disorders which can be treated on account of the tissue-relaxant action of the PDE inhibitors, such as, for example, erectile dysfunction or colics of the kidneys and of the ureters in connection with kidney stones. In addition, the compounds of the invention are useful in the treatment of diabetes insipidus, diabetes mellitus, leukaemia, osteoporosis and conditions associated with cerebral metabolic inhibition, such as cerebral senility, senile dementia (Alzheimer's disease), memory impairment associated with Parkinson's disease or multiinfarct dementia; and also illnesses of the central nervous system, such as depressions or arteriosclerotic dementia; as well as for enhancing cognition.

The invention further relates to a method for the treatment of mammals, including humans, which are suffering from one of the above mentioned illnesses. The method is characterized in that a therapeutically active and pharmacologically effective and tolerable amount of one or more of the compounds according to the invention is administered to the ill mammal.

The invention further relates to the compounds according to the invention for use in the treatment and/or prophylaxis of illnesses, especially the illnesses mentioned.

The invention further relates to the compounds according to the invention having PDE, particularly PDE4, inhibiting properties.

The invention also relates to the use of the compounds according to the invention for the production of pharmaceutical compositions which are employed for the treatment and/or prophylaxis of the illnesses mentioned.

The invention furthermore relates to pharmaceutical compositions for the treatment and/or prophylaxis of the illnesses mentioned, which contain one or more of the compounds according to the invention.

Additionally, the invention relates to an article of manufacture, which comprises packaging material and a pharmaceutical agent contained within said packaging material, wherein the pharmaceutical agent is therapeutically effective for antagonizing the effects of the cyclic nucleotide phosphodiesterase of type 4 (PDE4), ameliorating the symptoms of an PDE4-meditated disorder, and wherein the packaging material comprises a label or package insert which indicates that the pharmaceutical agent is useful for preventing or treating PDE4-meditated disorders, and wherein said pharmaceutical agent comprises one or more compounds of formula 1 according to the invention. The packaging material, label and package insert otherwise parallel or resemble what is generally regarded as standard packaging material, labels and package inserts for pharmaceuticals having related utilities.

The pharmaceutical compositions are prepared by processes which are known per se and familiar to the person skilled in the art. As pharmaceutical compositions, the compounds according to the invention (=active compounds) are either employed as such, or preferably in combination with suitable pharmaceutical auxiliaries and/or excipients, e.g. in the form of tablets, coated tablets, capsules, caplets, suppositories, patches (e.g. as TTS), emulsions, suspensions, gels or solutions, the active compound content advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries or excipients which are suitable for the desired pharmaceutical formulations on account of his/her expert knowledge. In addition to solvents, gel formers, ointment bases and other active compound excipients, for example antioxidants, dispersants, emulsifiers, preservatives, solubilizers, colorants, complexing agents or permeation promoters, can be used.

The administration of the pharmaceutical compositions according to the invention may be performed in any of the generally accepted modes of administration available in the art. Illustrative examples of suitable modes of administration include intravenous oral, nasal, parenteral, topical, transdermal and rectal delivery. Oral delivery is preferred.

For the treatment of disorders of the respiratory tract, the compounds according to the invention are preferably also administered by inhalation in the form of an aerosol; the aerosol particles of solid, liquid or mixed composition preferably having a diameter of 0.5 to 10 μm, advantageously of 2 to 6 μm.

Aerosol generation can be carried out, for example, by pressure-driven jet atomizers or ultrasonic atomizers, but advantageously by propellant-driven metered aerosols or propellant-free administration of micronized active compounds from inhalation capsules.

Depending on the inhaler system used, in addition to the active compounds the administration forms additionally contain the required excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of apparatuses are available with which aerosols of optimum particle size can be generated and administered, using an inhalation technique which is as right as possible for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhaler described in European Patent Application EP 0 505 321), using which an optimal administration of active compound can be achieved.

For the treatment of dermatosis the compounds according to the invention are in particular administered in the form of those pharmaceutical compositions which are suitable for topical application. For the production of the pharmaceutical compositions, the compounds according to the invention (=active compounds) are preferably mixed with suitable pharmaceutical auxiliaries and further processed to give suitable pharmaceutical formulations. Suitable pharmaceutical formulations are, for example, powders, emulsions, suspensions, sprays, oils, ointments, fatty ointments, creams, pastes, gels or solutions.

The pharmaceutical compositions according to the invention are prepared by processes known per se. The dosage of the actve compounds is carried out in the order of magnitude customary for PDE inhibitors. Topical application forms (such as ointments) for the treatment of dermatosis thus contain the active compounds in a concentration of, for example, 0.1-99%. The dose for administration by inhalation is customarly between 0.1 and 3 mg per day. The customary dose in the case of systemic therapy (p.o. or l.v.) is between 0.03 and 3 mg/kg per day.

Biological Investigations

The second messenger cyclic AMP (cAMP) is well-known for inhibiting inflammatory and immuno-competent cells. The PDE4 isoenzyme is broadly expressed in cells involved in the initiation and propagation of inflammatory diseases (H Tenor and C Schudt, in, Phosphodiesterase inhibitors”, 21 -40, “The Handbook of Immunopharmacology”, Academic Press, 1996), and its inhibition leads to an increase of the intracellular cAMP concentration and thus to the inhibition of cellular activation (JE Souness et al., Immunopharmacology 47:127-162. 2000).

The antiinflammatory potential of PDE4 inhibitors in vivo in various animal models has been described (M M Teixeira, TIPS 18:164-170, 1997). For the investigation of PDE4 inhibition on the cellular level (in vitro), a large variety of proinflammatory responses can be measured. Examples are the superoxide production of neutrophilic (C Schudt et al., Arch Pharmacol 344:682-690, 1991) or eosinophilic (A Hatzelmann et al., Brit J. Pharmacol 114:821-831, 1995) granulocytes, which can be measured as luminol-enhanced chemiluminescence, or the synthesis of tumor necrosis factor-α in monocytes, macrophages or dendritic cells (Gantner et al., Brit J. Pharmacol 121:221-231, 1997, and Pulmonary Pharmacol Therap 12:377-386, 1999). In addition, the immunomodulatory potential of PDE4 inhibitors is evident from the inhibition of T-cell responses like cytokine synthesis or proliferation (D M Essayan, Biochem Pharmacol 57:965-973, 1999). Substances which inhibit the secretion of the aforementioned proinflammatory mediators are those which inhibit PDE4. PDE4 inhibition by the compounds according to the invention is thus a central indicator for the suppression of inflammatory processes.

Method for Measuring Inhibition of PDE4 Activity

PDE4 activity was determined as described by Thompson et al. (Adv Cycl Nucl Res 10:69-92, 1979) with some modifications (Bauer and Schwabe, Naunyn-Schmiedeberg's Arch Pharmacol 311:193-198, 1980). At a final assay volume of 200 μl (96well microtiter plates) the assay mixture contained 20 mM Tris (pH 7.4), 5 mM MgCl₂, 0.5 μM cAMP, [³H]cAMP (about 30,000 cpm/assay), the test compound and an aliquot of cytosol from human neutrophilis which mainly contains PDE4 activity as described by Schudt et al. (Naunyn-Schmledeberg's Arch Pharmacol 344:682-690, 1991); the PDE3-specific inhibitor Motapizone (1 μM) was included to suppress PDE3 activity originating from contaminating platelets. Serial dilutions of the compounds were prepared in DMSO and further diluted 1:100 (v/v) in the assays to obtain the desired final concentrations of the inhibitors at a DMSO concentration of 1% (v/v) which by itself only slightly affected PDE4 activity.

After preincubation for 5 min at 37° C., the reaction was started by the addition of substrate (cAMP) and the assays were incubated for further 15 min at 37° C. 50 μl of 0.2 N HCl was added to stop the reaction and the assays were left on ice for about 10 min. Following incubation with 25 μg 5′-nucleotidase (Crotalus atrox snake venom) for 10 min at 37° C., the assays were loaded on QAE Sephadex A-25 (1 ml bed volume). The columns were eluted with 2 ml of 30 mM ammonium formulate (pH 6.0) and the was counted for radioactivity. Results were corrected for blank values (measured in the presence of denatured protein) which were below 5% of total radioactivity. The amount of cyclic nucleotides hydrolyzed did not exceed 30% of the original substrate concentration. The IC₅₀ -values for the compounds according to the invention for the inhibition of the PDE4 activity were determined from the concentration-inhibition curves by nonlinear-regression.

Representative inhibitory values determined for the compounds according to the inventon follow from following table A, in which the numbers of the compounds correspond to the numbers of the examples.

TABLE A
Inhibition of the PDE4 activity

| Compound | −log IC₅₀ |
|---|---|
| 1 | 9.43 |
| 2 | 9.04 |
| 4 | 8.20 |
| 5 | 8.41 |
| 6 | 8.08 |
| 7 | 8.26 |
| 8 | 8.01 |
| 11 | 8.87 |
| 12 | 8.48 |
| 13 | 8.41 |
| 14 | 8.43 |
| 15 | 8.33 |
| 16 | 8.36 |
| 17 | 8.47 |
| 18 | 8.73 |
| 19 | 8.73 |
| 20 | 8.00 |
| 22 | 8.62 |
| 23 | 8.75 |
| 24 | 8.25 |
| 25 | 8.33 |
| 26 | 9.27 |

1. A compound of the formula I, 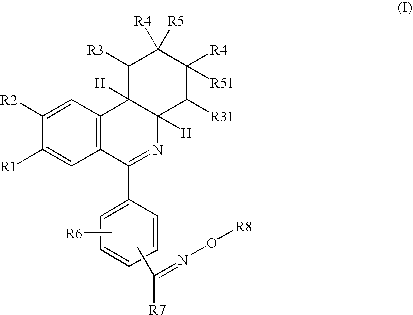
in which
R1 is hydroxyl, 1-4C-alkoxy, 3-7C-cycloalkoxy, 3-7C-cycloalkylmethoxy or completely or predominantly fluorine-substituted 1-4C-alkoxy,
R2 is hydroxyl, 1-4C-alkoxy, 3-7C-cycloalkoxy, 3-7C-cycloalkylmethoxy or completely or predominantly fluorine-substituted 1-4C-alkoxy,
or in which R1 and R2 together are a 1-2C-alkylenedioxy group,
R3 is hydrogen or 1-4C-alkyl,
R31 is hydrogen or 1-4C-alkyl, p1 or in which R3 and R31 together are a 1-4C-alkylene group,
R4 is hydrogen or 1-4C-alkyl,
R5 is hydrogen,
R51 is hydrogen,
or in which R5 and R51 together represent an additional bond,
R6 is hydrogen, halogen, nitro, 1-4C-alkyl, trifluoromethyl or 1-4C-alkoxy,
R7 is 1-4C-alkyl, 3-7C-cycloalkyl, 3-7C-cycloalkylmethyl, pyridinyl, phenyl or R71- and/or R72-substituted phenyl, wherein
R71 is halogen, hydroxyl, cyano, trifluoromethyl, carboxyl, nitro, 1-4C-alkyl or 1-4C-alkoxy,
R72 is 1-4C-alkoxy, 1-4C-alkyl or halogen,
R8 is hydrogen, phenyl, 1-4C-alkyl, aryloxy-2-4C-alkyl or R9-substituted 1-4C-alkyl, wherein
aryloxy is phenoxy or R81-substituted phenoxy, wherein
R81 is halogen or trifluoromethyl,
R9 is phenyl, C(O)N(R91)R92 or R93-substituted phenyl, wherein
R91 is phenyl, aryl-1-4C-alkyl or R911-substituted phenyl, wherein
aryl is phenyl or R81-substituted phenyl,
R911 is 1-4C-alkoxy or completely or predominantly fluorine-substituted 1-4C-alkoxy,
R92 is hydrogen,
or wherein R91 and R92, together and including the nitrogen atom to which both are bound, represent a 1-pyrrolidinyl, 1-piperidinyl, 1-piperazinyl, 4-methylpiperazin-1-yl, 4-phenylpiperazin-1-yl, 1-hexahydroazepinyl or 4-morpholinyl radical,
R93 is nitro, 1-4C-alkyl, 1-4C-alkoxycarbonyl, halogen, trifluoromethyl, 1-4C-alkoxy or completely or predominantly fluorine-substituted 1-4C-alkoxy,
or a salt or an E/Z isomer thereof.

2. A compound of the formula I as claimed in claim 1, in which
R1 is 1-2C-alkoxy, 3-5C-cycloalkoxy, 3-5C-cycloalkylmethoxy or completely or predominantly fluorine-substituted l-2C-alkoxy,
R2 is 1-2C-alkoxy, 3-5C-cycloalkoxy, 3-5C-cycloalkylmethoxy or completely or predominantly fluorine-substituted 1-2C-alkoxy,
R3 is hydrogen,
R31 is hydrogen,
R4 is hydrogen or 1-2C-alkyl,
R5 is hydrogen,
R51 is hydrogen,
or in which R5 and R51 together represent an additional bond,
R6 is hydrogen,
R7 is 1-4C-alkyl or phenyl,
R8 is hydrogen, phenyl, 1-4C-alkyl, aryloxy-2-4C-alkyl or R9-substituted 1-2C-alkyl, wherein
aryloxy is phenoxy or R81-substituted phenoxy, wherein
R81 is halogen or trifluoromethyl,
R9 is phenyl, C(O)N(R91)R92 or R93-substituted phenyl, wherein
R91 is aryl-1-2C-alkyl or R911-substituted phenyl, wherein
aryl is phenyl or R81-substituted phenyl,
R911 is 1-4C-alkoxy or completely or predominantly fluorine-substituted 1-2C-alkoxy,
R92 is hydrogen,
or wherein R91 and R92, together and including the nitrogen atom to which both are bound, represent a 4-methylpiperazin-1-yl, 4-phenylpiperazin-1-yl or 4-morpholinyl radical,
R93 is nitro, 1-4C-alkoxycarbonyl, halogen, trifluoromethyl, 1-4C-alkoxy or completely or predominantly fluorine-substituted 1-2C-alkoxy,
or a salt or an E/Z isomer thereof.

3. A compound of the formula I as claimed in claim 1, in which
R1 is methoxy,
R2 is methoxy,
R3, R31, R4, R5 and R51 are hydrogen,
R6 is hydrogen,
R7 is 1-2C-alkyl or phenyl,
R8 is hydrogen, phenyl, methyl, ethyl, isobutyl, aryloxyethyl or R9-substituted methyl, wherein
aryloxy is phenoxy or R81-substituted phenoxy, wherein
R81 is trifluoromethyl,
R9 is phenyl, C(O)N(R91)R92 or R93-substituted phenyl, wherein
R91 is arylmethyl or R911-substituted phenyl, wherein
aryl is chlorine-substituted phenyl,
R911 is methoxy,
R92 is hydrogen,
or wherein R91 and R92, together and including the nitrogen atom to which both are bound, represent a 4-phenylpiperazin-1-yl or 4-morpholinyl radical,
R93 is nitro, methoxycarbonyl, chloro, fluoro, trifluoromethyl or methoxy,
or a salt or an E/Z isomer thereof.

4. A compound of the formula I as claimed in claim 1, in which either
R1 is methoxy,
R2 is methoxy,
R3, R31, R4, R5 and R51 are hydrogen,
R6 is hydrogen,
R7 is methyl,
R8 is hydrogen, phenyl, methyl, ethyl, isobutyl, phenoxyethyl, 3-trifluoromethylphenoxyethyl or R9-substituted methyl, wherein
R9 is phenyl, 4-nitrophenyl, 4-methoxycarbonylphenyl, 4-chlorophenyl, 3-chlorophenyl, 2-chlorophenyl, 4-fluorophenyl, 3-fluorophenyl, 4-trifluoromethylphenyl, 3-trifluoromethylphenyl, 4-methoxyphenyl, 2-methoxyphenyl or C(O)N(R91)R92, wherein
R91 is 2-methoxyphenyl or 4-chlorobenzyl,
R92 is hydrogen,
or wherein R91 and R92, together and including the nitrogen atom to which both are bound, represent a 4-phenylpiperazin-1-yl or 4-morpholinyl radical;
or
R1 is methoxy,
R2 is methoxy,
R3, R31, R4, R5 and R51 are hydrogen,
R6 is hydrogen,
R7 is phenyl,
R8 is hydrogen;
or a salt or an E/Z isomer thereof

5. A compound of the formula I according to claim 1, in which the hydrogen atoms in positions 4a and 10b are in the cis position relative to one another, or a salt or an E/Z isomer thereof.

6. A compound of the formula I according to claim 1, which have with respect to the positions 4a and 10b the configuration shown in formula I*: 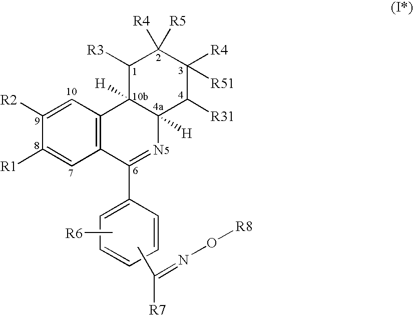
or a salt or an E/Z isomer thereof.

7. (canceled)

8. A pharmaceutical composition comprising one or more compounds of the formula I as claimed in claim 1 together with a pharmaceutically acceptable excipient or vehicle.

9. (canceled)

10. A method for treating a respiratory disorder or dermatoses in a patient comprising administering to said patient a therapeutically effective amount of a compound of the formula I as claimed in claim 1.

11. A method for treating an airway disorder in a patient comprising administering to said patient a therapeutically effective amount of a compound of the formula I as claimed in claim 1.
